(19) United States
(12) Patent Application Publication
(10) Pub. No.: US 2009/0023644 A1
(43) Pub. Date: Jan. 22, 2009

(54) Methods of using cgrp for cardiovascular and renal indications

(76) Inventors: Jeffery L. Southard, Olathe, KS (US); George Lee Southard, Sanibel, FL (US)

Correspondence Address: MORRISON & FOERSTER LLP, 755 PAGE MILL RD, PALO ALTO, CA, 94304-1018, US

(21) Appl. No.: 10/586,037

(22) PCT Filed: Jan. 13, 2005
(86) PCT No.: PCT/US05/01224

§ 371 Date: Jul. 13, 2006

Related U.S. Application Data
(60) Provisional application No. 60/608,945, filed on Jan. 13, 2004.

(51) Int. Cl. A61K 38/16; A61P 9/00

(52) U.S. Cl. 514/12

(57) ABSTRACT

The present invention provides methods of treating and preventing mortality associated with heart failure and renal insufficiency in a Stage B, C, or D heart failure patient, and for improving quality of life by providing improved methods of administering a therapeutically effective amount CGRP. One method comprise administering between about 50 and about 500 ng/min of CGRP for a time between 30 minutes and 8 hours per day for as many days as needed to provide symptomatic relief, prevent exacerbation of symptoms, and/or prevent and/or delay progression of the disease state of heart failure in said patient. The therapies can be administered on an outpatient or inpatient basis.

### 1. FIELD OF THE INVENTION

This invention relates generally to methods for treating heart failure and improving renal function, and more particularly is directed to the administration of CGRP for the treatment or prevention of heart failure and for improving renal function.

### 2. DESCRIPTION OF THE PRIOR ART

Heart failure is a complex clinical syndrome that can result from any structural or functional cardiac disorder that impairs the ability of the ventricle to fill with or eject blood, and the heart works less efficiently than it should. Heart failure is characterized by specific symptoms (e.g., dyspnea and fatigue) which may limit exercise tolerance and signs (e.g., fluid retention) which may lead to pulmonary congestion and peripheral edema. Both abnormalities can impair the functional capacity and quality of life of affected individuals, but they may not necessarily dominate the clinical picture at the same time. Because not all patients have volume overload at the time of initial or subsequent evaluation, the term “heart failure” is preferred over the older term “congestive heart failure.”

The clinical syndrome of heart failure may result from disorders of the pericardium, myocardium, endocardium, or great vessel. For example, common causes of heart failure include: narrowing of the arteries supplying blood to the heart muscle (coronary heart disease); prior heart attack (myocardial infarction) resulting in scar tissue large enough to interfere with normal function of the heart; high blood pressure; heart valve disease due to past rheumatic fever or an abnormality present at birth; primary disease of the heart muscle itself (cardiomyopathy); defects in the heart present at birth (congenital heart disease) and infection of the heart valves and/or muscle itself (endocarditis and/or myocarditis or pericarditis). The majority of patients with heart failure have symptoms due to an impairment of left ventricular function. Each of these disease processes can lead to heart failure by reducing the strength and efficiency of the heart muscle contraction, by limiting the ability of the heart's pumping chambers to fill with blood due to mechanical problems or impaired diastolic relaxation, or by filling the chambers with too much blood.

Renal blood flow is also an important factor in the development of the clinical syndrome of heart failure. It is a determinant of some important neurohormonal responses and of salt and water retention. Renal blood flow is reduced in patients with HF, and many patients with HF will also eventually develop renal failure.

There are four stages of heart failure recognized by the American College of Cardiology Guidelines for the Evaluation and Management of Chronic Heart Failure in the Adult. Stage A refers to patients who are at high risk for developing heart failure but have no identified structural or functional abnormalities of the heart and have never shown signs or symptoms of heart failure. If needed, Stage A patients are prescribed ACE inhibitors to lower blood pressure and reduce the heart's work load. Stage B refers to patients who have developed structural heart disease strongly associated with the development of heart failure but have never shown signs or symptoms of heart failure. Stage B patients are typically prescribed ACE inhibitors and beta-blockers that decrease myocardial oxygen demand and thereby ischemia, and reduce heart rate and cardiac work. Stage C refers to current or prior symptoms of heart failure associated with underlying structural disease. Management of HF at Stage C can involve a triple or quadruple drug therapy that includes ACE inhibitors, beta-blockers, diuretics, and Digitalis. Stage D refers to patients with advanced structural heart disease and marked symptoms of heart failure at rest despite maximal medical therapy, requiring specialized intervention. Since HF is a terminal condition, mid and end-stage HF (Stages C and D, respectively) treatment focuses on alleviating symptoms and increasing the patient's quality of life such that they can continue to live a relatively active lifestyle. Successful management of the progression of heart failure and effective treatments to relieve heart failure symptoms are determined by monitoring increases in the heart's ejection fraction, decreases in dyspnea, and changes in the frequency and/or severity of heart failure symptoms. However, while current end-stage drug therapies such as Dobutamine or Milrinone increase the patient's quality of life, they also have been shown to increase mortality.

It is estimated that about four million people in the United States suffer from various degrees of heart failure. Although heart failure is a chronic condition, the disease often requires acute hospital care. Patients are commonly admitted for acute pulmonary congestion accompanied by serious or severe shortness of breath. Acute care for HF accounts for the use of more hospital days than any other cardiac diagnosis, and consumes in excess of seven and one-half billion dollars in the United States annually.

Current research into the treatment of chronic heart failure is focused on providing cardioprotection, myocardial tissue salvage by minimizing or reducing infarction size, and preventing reperfusion injury. Many current drug therapies for treating heart failure address specific clinical aspects associated with myocardial infarction, such as antiplatelet/fibrinolytic, anti-inflammatory, and antioxidant activities. Such drugs include ACE inhibitors to prevent blood vessel constriction and to increase blood flow to the body, diuretics to remove excess fluid, beta blockers to reduce heart work load, calcium channel blockers to increase the blood flow through the heart and prevent vessel constriction, blood thinners to prevent blood clots, and cardiotonics to strengthen the heart's ability to pump blood. Only a few companies to date are developing new drugs that address tissue salvage, however the effectiveness of these drugs remains to be established in the clinic. As with all drugs, these agents must be taken in doses sufficient to ensure their effectiveness. Problematically, however, over-treatment can lead to hypotension, renal impairment, hyponatremia, hypokalemia, worsening heart failure, impaired mental functioning, and other adverse conditions. Surgical treatments include angioplasty, coronary arty by-pass grafts, valve replacement, pacemakers, internal defibrillators, left ventricular assist devices, and heart transplants.

Heart failure is the number one diagnosis for hospital admissions in patients over the age of 65. More than $38.1 billion has been spent annually since 1991 on inpatient and outpatient costs and greater than $500 million on drugs to treat HF. The disorder is the underlying reason for 12 to 15 million office visits each year and 1.7 to 2.6 million hospital admissions each year. Because of the hospitalization costs required to treat a heart failure patient, the current trend is to get HF patients into outpatient care as soon as possible, often within the 48 hours of hospital admission. Specialized outpatient clinics are now available for heart failure patients. The patients typically attend the clinic between one and four times per week to receive intravenous infusions of a prescribed heart failure therapy until hemodynamic symptoms improve.

Calcitonin gene-related peptide (“CGRP”) is a 37-amino acid neuropeptide which is the most potent naturally occurring vasodilator peptide in the human body. CGRP is distributed throughout the central and peripheral nervous systems, and is found in areas that are known to be involved in cardiovascular function (Wimalawansa, S., Critical Reviews in Neurobiology, 11:167-239 (1997)). Peripherally, CGRP is found in the heart, particularly in association with the sinoatrial and atrioventricular nodes. In addition, CGRP is found in nerve fibers that form a dense periadventitial network throughout the peripheral vascular system, including the cerebral, coronary, and renal arteries. CGRP has prominent cardiovascular effects, including vasodilation and positive chronotropic and inotropic effects, which may play an important role in normal cardiovascular function (Wimalawansa, S., Endocrine Reviews, 17:208:217 (1996)).

When administered, CGRP has pronounced cardiovascular benefits, including vasodilation, ischemic cardioprotection, reduction in infarction size due to heart attack, inhibition of platelet aggregation and smooth muscle cell proliferation which can potentially reduce the incidence of restenosis, increased renal function, and overall increased efficiency of cardiovascular functions. As a result of providing cardioprotection, minimizing reperfusion injury, and reducing infarction size, CGRP also promotes myocardial tissue salvage. CGRP also plays a role in regulating inotropy, chronotropy, microvascular permeability, vascular tone, and angiogenesis. CGRP also has significant advantages over conventional drug treatments. First, CGRP does not produce the potentially dangerous side effects, toxicity and tolerance associated with conventional cardiovascular drugs such as Nitroglycerin, Dobutamine and Natrecor. In fact, CGRP has been reported to down-regulate immune response via inhibition of cytokine release and has been safely administered to immuno-suppressed subjects without the induction of sensitivity. Second, because CGRP has multiple hemodynamic benefits, it can potentially reduce or eliminate the need for drug cocktails to maintain specific hemodynamic functions. Third, the biochemical activity of CGRP is mediated through specific receptor binding sites concentrated in the heart, kidneys, and genitilia, and is known to act on two specific CGRP receptor subtypes located on the surface of the endothelial and smooth muscle cells, respectively. Accordingly, CGRP exhibits virtually no side effects or tolerance when administered systemically.

Studies have demonstrated that acute administration of CGRP can result in increased cardiac performance and reduced systemic resistance in a number of clinical scenarios. For example, Anand, et al. (J. Am. Coll. Cardiol., 17:208-217 (1991)) reported that short-term IV infusions (10 or 20 minutes) of CGRP at rates of 0.8, 3.2, or 16 ng/kg/min (i.e., 56, 224, or 1120 ng/min based on a 70 kg subject) produced beneficial hemodynamic effects such as decreased systemic vascular resistance and increase in cardiac output, with no tachycardia observed. The study concluded that at lower doses CGRP behaves as a pure arteriolar vasodilator, where as at the higher dose CGRP acts a mixed vasodilator. Stephenson, et al. (Int. J. Cardiol., 37:407-414 (1992)) reported administration of CGRP at a rate of 600 ng/min by either a 48-hour continuous IV infusion or 2-8 hour infusions for two consecutive days. In the continuous infusion therapy, infusion was discontinued after 28 hours in 3 out of the 6 patients due to nausea, diarrhea, and/or severe facial flushing. On the other hand, the pulsed therapy was well tolerated and was observed to improve hemodynamic functions such as left ventricular function. However, unfavorable side effects of tachycardia and neurohumoral response were also observed with the pulsed therapy. Sekhar, et al. (Am. J. Cardiol. 67:732-736 (1991) reported administration of CGRP at a rate of 8 ng/kg/min (i.e., 560 ng/min based on a 70 kg subject) by IV infusion for 8 hours. This therapy was observed to have beneficial hemodynamic effects such as decreased pulmonary and systemic arterial pressure, decreased vascular resistance and increased cardiac output. It was also observed that renal blood flow and glomerular filtration were increased during treatment. However, the hemodynamic effects were lost within 30 minutes of stopping CGRP infusion.

Chronic HF is a progressive disease. Therefore, therapies that initially seek to reduce disease progression while increasing the patient's quality of life and relieving symptoms that exacerbate the condition are desirable. It would be far more cost effective and much better for the patient's health if chronic heart failure could be managed and controlled by the routine or controlled release administration of appropriate drug therapy rather than by hospital treatment upon the manifestation of acute symptoms.

### SUMMARY OF THE INVENTION

The present invention provides methods for the treatment or prevention of heart failure (“HF”) and/or rental failure in patients comprising novel methods of administering CGRP. The methods include prolonged CGRP dosing regimes for HF patients which treat the conditions underlying HF while minimizing or attenuating the deleterious effects commonly associated with CGRP such as nausea, diarrhea, severe facial flushing and. intermittent tachycardia.

More specifically, one aspect of this invention provides a method, of treating HF and/or renal failure in a patient comprising administering CGRP to the patient at a rate between about 50 and 500 ng/min for a time between about 30 minutes and 8 hours per day for as many days as needed to provide symptomatic relief, prevent exacerbation of symptoms, and/or prevent and/or delay progression of the disease state of heart failure in the patient.

This invention further provides a method of treating HF and/or renal failure in a patient comprising administering CGRP to the patient at a rate between about 500 and 600 ng/min for up to 8 hours per day for at least three consecutive days or several times per week as needed to provide symptomatic relief, prevent exacerbation of symptoms, and/or prevent and/or delay progression of the disease state of HF in the patient.

Another aspect of this invention provides a method of treating HF and/or renal failure in a patent, comprising administering CGRP to a heart failure patient as an initial or maintenance therapy at a rate between 0.8 to 10 ng/kg/min over a period of 1 to 8 hours two or more times per day as needed to provide symptomatic relief, prevent exacerbation of symptoms, and/or prevent and/or delay progression of the disease state of heart failure in said patient.

The above-described methods can further be used as maintenance therapy, preferably at lower doses or dosing rates of CGRP after the initial therapy is completed.

This invention further provides prophylactic methods for preventing HF in a patient at risk for HF or slowing the progression or symptoms of HF in a patient suffering from HF. For example, another aspect of this invention provides a method of preventing or reducing the risk of occurrence of myocardial infarction in a patient, comprising administering to a human at risk of having a myocardial infarction a CGRP formulation in an amount effective to prevent or reduce the risk of said myocardial infarction.

In all of the above-described methods, the amount of CGRP delivered to the patient depends on the symptoms, stage of HF, degree of severity and/or other medications (e.g., diuretics) being administered to the patient.

This invention further provides a method of augmenting current HF therapies comprising administering CGRP according to the dosing regimes of this invention together with one or more addition drugs for HF, wherein CGRP and the additional drug(s) can be administered together, separately and simultaneously, or separately in any order.

This invention further provides a method of counteracting ischemia in a patient, wherein the ischemia is due to a myocardial infarction, the method comprising administering CGRP to the patient as an initial or maintenance therapy, alone or in conjunction with other interventional therapies, at a rate between 0.8 to 16 ng/kg/min for up to 24 hours per day as needed to provide cardioprotection, reduction in infarction size, reduction in reperfusion injury, symptomatic relief, and/or prevent exacerbation of symptoms.

Another aspect of this invention comprises a method of improving renal blood flow and glomerular filtration in a patient suffering from diminished renal function, comprising administering CGRP to a patient in need thereof in a manner effective to improve renal blood flow and/or glomerular filtration.

In any of the methods described herein, CGRP or a pharmaceutically acceptable formulation thereof can be administered by many known methods of drug administration, including, but not limited to, parenteral, transdermal or mucosal administration.

Administration of CGRP according to the methods of this invention provides a safer and more effective treatment of acute cardiac ischemia and heart failure compared to current treatments for HF. Given the advantages in cardioprotection, myocardial tissue salvage, cardiac hemodynamic improvement, and renal function upon treatment with CGRP, the methods of this invention have the potential to be powerful frontline weapons in the arsenal of an emergency room doctor who is the first to treat patients suffering from myocardial infarction (MI) upon entry into the health care system, and/or an interventional cardiologist who is working to re-establishing blood flow to an ischemic heart using angioplasty or stenting procedures, and/or a cardiologist who is treating mid- to end-stage HF patients to provide increased quality of life to terminal patients.

Additional advantages and novel features of this invention shall be set forth in part in the description that follows, and in part will become apparent to those skilled in the art upon examination of the following specification or may be learned by the practice of the invention. The advantages of the invention may be realized and attained by means of the instrumentalities, combinations, compositions, and methods particularly pointed out in the appended claims.

### DETAILED DESCRIPTION OF THE INVENTION

One aspect of this invention provides improved methods for administering CGRP to a patient having HF in a manner effective to treat or prevent HF. The treatment according to any of the methods of this invention can be administered on an inpatient such as a hospital or emergency room, or in an outpatient setting such as a hospice or home health care setting or administration by emergency care personnel to a patient having a myocardial infarction. This invention further provides methods of attenuating symptoms of HF and/or improving hemodynamic functions in a patient with HF by providing improved methods of administering CGRP to the patient in either an inpatient or outpatient setting.

“Treating HF” as used herein refers to treating any one or more of the conditions underlying HF, including, without limitation, decreased cardiac contractility, abnormal diastolic compliance, reduced stroke volume, pulmonary congestion, decreased cardiac output, and other diminished hemodynamic functions, while minimizing or attenuating deleterious effects that may be associated with the long-term administration of CGRP such as nausea, diarrhea, severe facial flushing and intermittent tachycardia. “Treating HF” also includes relieving or attenuating symptoms associated with HF.

This invention also provides a method of improving the quality of life in a patient with HF. “Quality of life” refers to one or more of a person's ability to walk, climb stairs, do errands, work around the house, participate in recreational activities, and/or not requiring frequent rest intermittently during activities, and/or the absence of sleeping problems or shortness of breath.

For purposes of this invention, a “patient having HF” refers to a person having Stage B, Stage C, or Stage D heart failure as classified in the American College of Cardiology Guidelines for the Evaluation and Management of Chronic Heart Failure in the Adult. While the American College of Cardiology Guidelines excluded HF in children, for purposes of this invention the methods are to be considered applicable, to any patient, regardless of age.

More specifically, this invention provides improved methods for providing effective amounts of CGRP for treating or preventing HF and/or for improving renal function in a patient. In the treatment of HF according to this invention, compositions comprising CGRP alone or in combination with other drugs or therapies will be formulated, dosed, and administered in a fashion consistent with good medical practice. It is to be understood that the actual dose will depend on the particular factors of each case. Generally, the dosage required to provide an effective amount of CGRP or pharmaceutically acceptable formulations thereof are in the ranges disclosed herein and can be adjusted by one of ordinary skill in the art. The dosage will vary depending on the clinical condition of the individual patient (especially the side effects of treatment with CGRP alone or in combination with other therapeutics), the age, health, physical condition, sex, diet and medical condition of the patient, the severity (i.e., stage) of heart failure, the route of administration, the site of delivery of CGRP, the type of drug delivery system that is used, whether CGRP is administered as part of a drug combination, the scheduling of administration, and other factors known to practitioners. Thus, while individual needs may vary, determination of optimal ranges for effective amounts of CGRP (alone or in combination with other drugs) within the ranges disclosed herein is within the expertise of those skilled in the art. Accordingly, “effective amounts” of each component for purposes herein are determined by such considerations and are amounts that improve one or more hemodynamic functions and/or ameliorate on or more deleterious conditions in HF patients and/or improve the quality of life in HF patients and/or improve renal function.

The term “hemodynamic functions” includes, but is not limited to, heart rate, right atrial pressure, pulmonary artery pressure, pulmonary artery wedge pressure, systemic arterial pressure, cardiac output (i.e., cardiac index), stroke volume index, pulmonary vascular resistance, and systemic vascular resistance.

The term “improved hemodynamic functions” includes, but is not limited to, increased cardiac output, decreased pulmonary artery wedge pressure, decreased pulmonary vascular resistance, and decreased systemic vascular resistance, increased cardiac contractility, normal diastolic compliance, increased stroke volume and reduced pulmonary congestion.

The term “afterload” refers to the resistance that the heart has to overcome during every beat to send blood into the aorta. This resistive force includes vasoactivity and blood viscosity.

The term “cardiac index” (CI) refers to amount of blood pumped by the heart per minute per meter squared of body surface area.

The term “cardiac output” (CO) refers to the volume of blood pumped by the heart in one minute. Increased cardiac output can indicate a high circulating volume. Decreased cardiac output indicates a decrease in circulating volume or a decrease in the strength of ventricular contraction.

The term “central venous pressure” (CVP) refers to readings that are used to approximate the Right Ventricular End Diastolic Pressure (RVEDP). The RVEDP assesses right ventricular function and general fluid status. Low CVP values typically reflect hypovolemia or decreased venous return, and high CVP values reflect overhydration, increased venous return or right-sided cardiac failure.

The term “change in heart rate” refers to a condition that indicates tachycardia or increased workload.

The term “dyspnea” means shortness of breath. Dyspnea is a primary clinical endpoint to address efficacy in heart failure treatments.

The term “left ventricular stroke index” (LVSI) refers to the difference in contractile position of the left ventricle from the resting position to the point of maximum contraction.

The term “mean arterial pressure” (MAP) refers to changes in the relationship between cardiac output (CO) and systemic vascular resistance (SVR) and reflects the arterial pressure in the vessels perfusing the organs. A low MAP indicates decreased blood flow through the organs, and a high MAP indicates an increased cardiac workload.

The term “neurohormone release” refers to a response by the kidneys to increase renal blood flow by releasing the vasoconstricting neurohormones norepinephrine, epinephrine, and rennin. These hormones act to constrict peripheral vasculature adversely affecting the PVR.

The term “preload” refers to the combination of pulmonary blood filling the atria and the stretching of myocardial fibers. Preload is regulated by the variability in intravascular volume. A reduction in volume decreases preload, whereas an increase in volume increases preload, mean arterial pressure (MAP) and stroke index (SI). Preload occurs during diastole.

The term “pulmonary artery pressure” (PA pressure) refers to blood pressure in the pulmonary artery. Increased pulmonary artery pressure may indicate a left-to-right cardiac shunt, pulmonary artery hypertension, COPD, emphysema, pulmonary embolus, pulmonary edema, or left ventricular failure.

The term “pulmonary capillary wedge pressure” (PCWP or PAWP) refers to a pressure are used to approximate LVEDP (left ventricular end diastolic pressure). High PCWP may indicate left ventricle failure, mitral valve pathology, cardiac insufficiency, and/or cardiac compression post hemorrhage. PCWP is a primary clinical endpoint to address efficacy in heart failure treatments.

The term “pulmonary vascular resistance” (PVR) refers to the measurement of resistance or the impediment of the pulmonary vascular bed to blood flow. An increased PVR is caused by pulmonary vascular disease, pulmonary embolism, pulmonary vasculitis, or hypoxia. A decreased PVR is caused by medications such as calcium channel blockers, aminophylline or isoproterenol, or by the delivery of O₂.

The term “renal blood flow” (RBF) refers to the measurement of blood flow into the kidneys. Twenty percent of cardiac output passes through the kidneys, which compromise less than 1% of body weight. Increased renal blood flow is proportional to increased renal function and urine output.

The term “renal glomerular filtration” (RBF) refers to the first step in urine formation as protein-free ultrafiltrate plasma crosses the walls of the glomerular capillaries. Increased renal blood flow increases flow of plasma across the glomeruli, increasing urine output.

The term “right ventricular pressure” (RV Pressure) refers to a direct measurement that indicates right ventricular function and general fluid status. High RV pressure may indicate pulmonary hypertension, right ventricle failure, or congestive heart failure.

The terms “stroke index” or “stroke volume index” (SI or SVI) are used interchangeably and refer to the amount of blood ejected from the heart in one cardiac cycle, relative to Body Surface Area (BSA). SVI is measured in milliliters per meter squared per beat. An increased SVI can be indicative of early septic shock, hyperthermia or hypervolemia, or can be caused by medications such as dopamine, dobutamine or digitalis. A decreased SVI can be caused by CHF, late septic shock, beta-blockers or an MI.

The term “stroke volume” (SV) refers to the amount of blood pumped by the heart per cardiac cycle, and is measured in milliliters per beat. A decreased SV may indicate impaired cardiac contractility or valve dysfunction and may result in heart failure. An increased SV can be caused by an increase in circulating volume or an increase in inotropy.

The term “systemic vascular resistance” (SVR refers to the measurement of resistance or impediment of the systemic vascular bed to blood flow. An increase in SVR can be caused by vasoconstrictors, hypovolemia or late septic shock. A decrease in SVR can be caused by early septic shock, vasodilators, morphine, nitrates or hypercarbia.

A “microgram” (μg) is 1 millionth of a gram, i.e., 10⁻⁶ grams.

A “nanogram” (ng) is 1 billionth of a gram, i.e., 10⁻⁹ grams.

A “picogram” (pg) is 1 trillionth of a gram, i.e., 10⁻¹² grams.

Table 1 provides normal values for the above-described hemodynamic parameters.

TABLE 1

| Hemodynamic Parameter | Normal Value |
|---|---|
| Blood Pressure Systolic (SBP) | 90-140 mm Hg |
| Diastolic (DBP) | 60-90 mm Hg |
| Mean Arterial Pressure (MAP) | 70-100 mm Hg |
| Cardiac Index (CI) | 2.5-4 L/min/m² |
| Cardiac Output (CO) | 4-8 L/min |
| Central Venous Pressure (CVP) | 2-6 mm Hg |
| Pulmonary Artery Pressure (PA) | Systolic 20-30 mm Hg |
| | (PAS) |
| | Diastolic 8-12 mm Hg |
| | (PAD) |
| | Mean 25 mm Hg |
| | (PAM) |
| Pulmonary Capillary Wedge Pressure (PWCP) | 37-250 dynes/sec/cm³ |
| Right Ventricular Pressure (RV) | Systolic 20-30 mm Hg |
| | Diastolic 0-5 mm Hg |
| Stroke Index (SI) | 25-45 mL/m² |
| Systemic Vascular Resistance (SVR) | 800-1200 dynes/sec/ |
| | cm³ |

Various sources of CGRP may be used in the methods of this invention. For example, synthetic CGRP may be obtained using an automatic peptide synthesizer according to well known methods. One method for synthesizing the CGRP is the well known Merrifield method (see, Merrifield, R. B., J. Am. Chem. Soc., 85:2149 (1963) and Merrifield, R. B., Science, 232:341 (1986), which are specifically incorporated herein by reference). Human CGRP also may be obtained from commercial sources, such as Peninsula Laboratory (Belmont, Calif.), Bachem Biosciences, Inc. (King of Prussia, Pa.) and Sigma Chemicals (St. Louis, Mo.). Commercial grade human CGRP is not marketed for human use (since this grade is not GMP/GLP grade); therefore, commercially available human CGRP may be used in the present invention only if it is purified and sterilized so that it is fit for human use. Genetically engineered human CGRP can also be used in the present invention. Similar results also could be achieved using a CGRP analogue or an analogue based on the CGRP “receptor structure.” These include peptide-based analogues, as well as peptide-mimetic analogues. Accordingly, analogs that function, similarly to CGRP are considered to be equivalents of CGRP for purposes of this invention. CGRP derived from animals is biologically active and thus could also be used in the present invention; however, as a practical matter, animal-derived CGRP presents allergy and autoimmune problems and therefore is preferably avoided.

Other forms of CGRP which are suitable for use in the methods of this invention are pharmaceutically acceptable prodrugs of CGRP. A “pharmaceutically acceptable prodrug” is a compound that may be converted under physiological conditions or by solvolysis to the specified compound or to a pharmaceutically acceptable salt of such compound. Prodrugs of CGRP may be identified using routine techniques known in the art. Prodrugs include compounds wherein an amino acid residue, or a polypeptide chain of two or more (e.g., two, three or four) amino acid residues is covalently joined through an amide or ester bond to a free amino, hydroxy or carboxylic acid group of compounds of the present invention. Additional types of prodrugs are also encompassed. For instance, free carboxyl groups can be derivatized as amides or alkyl esters. Free hydroxy groups may be derivatized using groups including but not limited to hemisuccinates, phosphate esters, dimethylaminoacetates, and phosphoryloxymethyloxycarbonyls, as outlined in Advanced Drug Delivery Reviews 1996, 19, 115. Carbamate prodrugs of hydroxy and amino groups are also included, as are carbonate prodrugs, sulfonate esters and sulfate esters of hydroxy groups. Derivitization of hydroxy groups as (acyloxy)methyl and (acyloxy)ethyl ethers wherein the acyl group may be an alkyl ester, optionally substituted with groups including but not limited to ether, amine and carboxylic acid functionalities, or where the acyl group is an amino acid ester as described above, are also encompassed. Prodrugs of this type are described in J. Med. Chem. 1996, 39, 10. Free amines can also be derivatized as amides, sulfonamides or phosphonamides. All of these prodrug moieties may incorporate groups including but not limited to ether, amine and carboxylic acid functionalities. Other examples of such prodrug derivatives are described in a) Design of Prodrugs, edited by H. Bundgaard, (Elsevier, 1985) and Methods in Enzymology, Vol. 42, p. 309-396, edited by K. Widder, et al. (Academic Press, 1985); b) A Textbook of Drug Design and Development, edited by Krogsgaard-Larsen and H. Bundgaard, Chapter 5 “Design and Application, of Prodrugs”, by H. Bundgaard p. 113-191 (1991); c) H. Bundgaard, Advanced Drug Delivery Reviews, 8:1-38 (1992); d) H. Bundgaard, et al., J. Pharmaceutical Sciences, 77:285 (1988); and e) N. Kakeya, et al., Chem. Pharm. Bull., 32:692 (1984), each of which is specifically incorporated herein by reference.

When administered in controlled dosages, CGRP has pronounced cardiovascular benefits, including vasodilation, ischemic cardioprotection, reduction in infarction size due to heart attack, inhibition of platelet aggregation and smooth muscle cell proliferation to potentially reduce the incidence of restenosis, increased renal function, and overall increased efficiency of cardiovascular functions. CGRP also plays a role in regulating inotropy, chronotropy, microvascular permeability, vascular tone, and angiogenesis.

As stated, CGRP offers significant advantages over conventional drug treatments. First, CGRP does not produce the potentially dangerous side effects, toxicity and tolerance associated with conventional cardiovascular drugs such as Nitroglycerin, Dobutamine and Natrecor. In fact, CGRP has been reported to down-regulate immune response via inhibition of cytokine release and has been safely administered to immunosuppressed subjects without the induction of sensitivity. Second, since CGRP possesses multiple hemodynamic benefits, it potentially reduces or eliminates the need for drug cocktails to maintain specific hemodynamic functions. In fact, more than 20 years of research on the potency, safety and efficacy of the drug in animals and humans has demonstrated the cardiovascular benefits of CGRP and have shown that CGRP exhibits virtually no side effects or tolerance when administered systemically.

In general, there are four goals in treating HF patients (1) treating the symptoms, (2) slowing the progression of cardiac dysfunction, (3) decreasing length of hospital stay, and (4) increasing the time between hospitalization, all while minimizing health care costs. It is believed that the methods of treating HF according to this invention will achieve one or more of these goals.

According to one embodiment, this invention provides a method of treating HF in a patient comprising administering CGRP or a pharmaceutically acceptable composition thereof to the patient at a rate between about 50 and 500 ng/min for a time between 30 minutes and 8 hours per day for as many days as needed to provide symptomatic relief, prevent exacerbation of symptoms, and/or prevent and/or delay progression of the disease state of heart failure in said patient. For example, CGRP may be continuously or intermittently administered for a period of time between about 24 and 48 hours, or as a bolus dose. If CGRP is administered two or more times intermittently each day, lower doses, e.g., 0.8 to 10 ng/min can be administered.

Treatment is continued as needed to provide symptomatic relief, prevent exacerbation of symptoms, and/or prevent and/or delay progression of the disease state of heart failure in said patient, or until it is no longer well tolerated by the patient, or until a physician terminates treatment. For example, a physician may monitor one or more symptoms of HF, renal blood flow, glomerular filtration rates, and/or serum levels of urea and creatinine in a patient being treated with CGRP according to this invention and, upon observing attenuation of one or more symptoms of HF for a period of time, conclude that the patient can sustain the positive effects of the above-described treatment without further administration of CGRP for a period of time. If necessary, the patient may then return at a later point in time for additional treatment as needed.

According to another embodiment, this invention provides a method of treating HF in a patient comprising administering CGRP to the patient at a rate between about 500 and 600 ng/min for period between about 8 hours per day for at least three consecutive days or several times per week as needed to provide symptomatic relief, prevent exacerbation of symptoms, and/or prevent and/or delay progression of the disease state of heart failure in the patient. This treatment may be provided as outpatient therapy to prevent exacerbation of the heart failure and to enhance the quality of life in the patient.

As used herein, “day” means a 24-hour period. Thus' for example, “for at least three consecutive days” means for at least a 72-hour period. During or after the treatment, a physician may monitor one or more symptoms of HF, renal blood flow, glomerular filtration rates, and/or serum levels of urea or creatinine in the patient and, upon observing an improvement in one or more of the parameters for a period of time, conclude that the patient can sustain the positive effects of the treatment without further administration of CGRP for a period of time.

According to another embodiment, this invention provides a method of treating HF in a patient comprising administering CGRP to the patient at a rate between about 50 and 400 ng/min over a period of up to 8 hours per day for each day of hospitalization of the patient or as needed. In certain cases the patient may require higher doses, for example up to 2 μg/min over the same time period.

Once treatment with CGRP according to any of the methods of this invention has achieved the desired results, e.g., symptomatic relief, prevent exacerbation of symptoms, and/or prevent and/or delay progression of the disease state of heart failure, the patient can then receive maintenance therapy if desired. For example, a lower dose of CGRP, e.g., less than 10 ng/min, can be administered to the patient for maintenance therapy by any suitable route including, but not limited to, injection, intravenous administration, etc. In one embodiment, the delivery regime can be designed to deliver between CGRP at a rate between about 0.8 to 10 ng/min for a desired period of time, such as over a period of 3, 6 or 9 months.

Because CGRP therapy according to any of the methods of this invention prevents further damage from ischemic injury and promotes the healing process, it can also be used to delay or preclude further exacerbation of a heart condition into a more serious and progressive diseases such as HF. Thus, each of the above-described methods may also be used as a prophylactic treatment to prevent or slow the progression of early stages of HF to more advanced stages. That is, once treatment with CGRP according to any of the methods of this invention has achieved the desired results, the patient can optionally receive maintenance therapy thereafter. For example, one embodiment of this invention for providing maintenance therapy to a patient with a heart condition comprises providing a lower dose of CGRP, e.g., less than 10 ng/min, to the patient for maintenance therapy by any suitable route including, but not limited to, injection, intravenous administration, controlled release administration, etc. In one embodiment, the delivery system can be designed to deliver between CGRP at a rate between about 0.8 to 10 ng/min for a desired period of time, such as over a period of 3, 6 or 9 months. In an alternative embodiment, the patient can receive long-term, low dose, maintenance administration of CGRP from a controlled release formulation.

In addition, it is known that a patient that has suffered a myocardial infarction (MI) will likely suffer another MI in the future. Thus, a patient having an MI can be treated with an initial dose of CGRP according to any of the methods of this invention until one or more symptoms of HF has diminished, and subsequently can be put on a CGRP maintenance dosing regime. The maintenance regime can also be given to a post-MI patient that was initially treated for MI by means other than CGRP, and can also be used for HF patients that have not yet suffered an MI as a means to slow the progression of HF into the more advanced stages or to prevent or reduce the risk of MI in patients with advanced HF.

This invention further provides methods for improving renal function in a patient suffering from diminished renal function comprising administering CORP according to any of the above-describe dosing regimes for treating HF. As used herein, the term “improved renal function” includes increased glomerular filtration, increased renal blood flow and decreased serum levels of urea and creatinine.

If necessary, CGRP can be administered according to the methods of this invention either alone or in combination with at least one other agent including, but not limited to, anti-proliferative agents, anti-clotting agents, vasodilators, diuretics, beta-blockers, calcium ion channel blockers, blood thinners, cardiotonics, ACE inhibitors, anti-inflammatories, antioxidants, and/or gene therapeutics. When used in combination with other agents, CGRP and the agent can be administered separately (either simultaneously or separately in any order) or in admixture. In one embodiment, when CGRP and at least one other agent are administered as separate components, they are administered to the patient at about the same time. “About the same time” means that within about thirty minutes of administering one compound (e.g., CGRP) to the patient, the other compound (e.g., an anti-proliferative or anti-clotting agent) is administered to the patient. “About the same time” also includes concomitant or simultaneous administration of the compounds.

### Augmentation of Current HF Therapies

A further aspect of this invention provides a method of treating HF by administering CGRP according to any of the methods disclosed herein to augment current HF therapies. CGRP can be administered according to any of the dosing regimes of this invention together with one or more addition drugs for HF, wherein CGRP and the additional drug(s) can be administered together, separately and simultaneously, or separately in any order.

Acute Myocardial Infarction

In the treatment of acute MI, physicians take aggressive action to restore blood flow to the heart to minimize permanent ischemic damage. These treatments take the form of vasodilators (nitroglycerin) and antithrombolytics (streptokinase, tPA), and platelet aggregation inhibitors (gpIIb/IIIa) in the attempt to dilate the coronary arteries and dissolve the thrombus, and inhibit platelet aggregation. If treatment is successful in restoring blood flow, the patient may be sent to recover in the CCU or go to the catheterization lab for an angioplasty or stenting procedure to open any remaining occlusions. However, the ischemic event itself causes generation of free radicals, and this process is potentiated when the vessels are re-opened and blood flow restored, which results in further tissue damage. In this setting, CGRP therapy administered alone or in conjunction with other therapeutic interventions according to any of the methods of this invention, particularly the infusion methods, would augment the current therapies such as antithrombolytics by elevating the therapeutic benefits of these drugs. The cardioprotective benefits of CGRP when infused at the initial stages of evaluation and treatment would provide levels of CGRP suitable to minimize reperfusion injury when interventional therapy is initiated, and thus maximize positive acute and long-term recovery outcomes.

Accordingly, a further aspect of this invention comprises a method of counteracting ischemia in a patient wherein the ischemia is due to a myocardial infarction (MI). Thus, the patient need not be a HF patient but rather a person suffering from or at risk from suffering an MI. The method comprises administering. CGRP to the patient as an initial or maintenance therapy, alone or in conjunction with other interventional therapies, at a rate between 0.8 to 16 ng/kg/min for up to 24 hours per day as needed to provide cardioprotection, reduction in infarction size, reduction in reperfusion injury, symptomatic relief, and/or prevent exacerbation of symptoms.

Percutaneous Translumenal Coronary Angioplasty (PTCA) and Stenting

If antithrombolytic therapy is ineffectual in the emergency room, or if it is determined that elective PTCA intervention is required to restore blood flow, CGRP infusion therapy already in process in the emergency room or started in the catheterization lab would provide the same reperfusion benefits as those described above when blood flow is restored to the ischemic tissues. Additional benefits in the catheterization lab would be realized when CGRP infusion therapy locally dilates coronary blood vessels, decreases the incidence of vasospasms and no-reflow during procedures, increases renal blood flow, and assists in preventing platelet aggregation and smooth muscle cell proliferation at the acute time points (<24 hours) following PTCA. Currently, Reopro® or Integrillin® is administered in advance or during PTCA procedures to halt platelet aggregation and reduce the incidence of restenosis in the long-term (>48 hours). CGRP infusion therapy would augment these current restenosis therapies by elevating the therapeutic benefits of preventing reperfusion injury, as well as inhibiting platelet aggregation and smooth muscle cell proliferation in the acute-term (<24 hours).

Coronary Artery Bypass Surgery (CABG)

Whether CABG is performed as an emergency procedure or as elective surgery, CGRP infusion therapy would provide all of the benefits stated above with respect to acute MI treatment and PTCA procedures. As a result, a CABG procedure could potentially experience even great positive outcomes and fewer acute-term complications.

Coronary Care Unit (CCU) Recovery

CGRP infusion therapy in CCU patients would maximize the ability of CGRP to reduce infarction size and promote cardiac tissue salvage. Whether the therapy was initiated in the emergency room, the cauterization lab, the operating room, or the CCU, recovery and healing process will begin in the CCU where CGRP can be administered over the course of several days, and the long-term benefits of CGRP infusion therapy will realized.

In order to use CGRP for the therapeutic treatment (including prophylactic treatment) of mammals including humans according to the methods of this invention, it is normally formulated in accordance with standard pharmaceutical practice as a pharmaceutical composition. According to this aspect of the invention there is provided a pharmaceutical composition comprising CGRP in association with a pharmaceutically acceptable diluent or carrier, wherein the CGRP is present in an amount for effective treating or preventing HF and/or for improving renal function.

CGRP can be administered to a patient by any available and effective delivery system including, but not limited to, parenteral, transdermal, intranasal, sublingual, transmucosal, intra-arterial, or intradermal modes of administration in dosage unit formulations containing conventional nontoxic pharmaceutically acceptable carriers, adjuvants, and vehicles as desired, such as a depot or a controlled release biodegradable polymer.

More specifically, CGRP or a pharmaceutically acceptable formulation thereof may be formulated for parenteral administration, e.g., for intravenous, subcutaneous, or intramuscular injection. For an injectable formulation, a dose of CGRP may be combined with a sterile aqueous solution which is preferably isotonic with the blood of the patient. Such a formulation may be prepared by dissolving a solid active ingredient in water containing physiologically-compatible substances such as sodium chloride, glycine, and the like, and having a buffered pH compatible with physiological conditions so as to produce an aqueous solution, and then rendering the solution sterile by methods known in the art. The formulations may be present in unit or multi-dose containers, such as sealed ampules or vials. The formulation may be delivered by any mode of injection, including, without limitation, epifascial, intracutaneous, intramuscular, intravascular, intravenous, parenchymatous, subcutaneous, oral or nasal preparations (see, for example, U.S. Pat. No. 5,958,877, which is specifically incorporated herein by reference).

Pharmaceutical compositions may also be in the form of a sterile injectable aqueous or oily suspension, which may be formulated according to known procedures using one or more appropriate dispersing or wetting agents and suspending agents. A sterile injectable preparation may also be a sterile injectable solution or suspension in a non-toxic parenterally-acceptable diluent or solvent, for example a solution in 1,3-butanediol.

In another embodiment, CGRP can be suitably administered using an implantable pump, which is particularly applicable for outpatient treatment. For example, a constant rate pump may be used to provide a constant, unchanging delivery of CGRP over a period of time. Alternatively, a programmable pump may be used if changes to the infusion rate are desired. Constant rate and programmable pumps are well know in the art and need not be described further. CGRP may also be released or delivered from an implantable osmotic mini-pump such as that described in U.S. Pat. Nos. 5,728,396 and 6,358,247, the disclosures of which are specifically incorporated herein in their entirety. The release rate from an osmotic mini-pump may be modulated with a microporous, fast-response gel disposed in the release orifice .for controlled release or targeted delivery of CGRP. Osmotic pumps are preferred in that they are much smaller than the constant rate and programmable pumps.

CGRP may also be administered to a patient via transdermal delivery devices, patches, electrophoretic devices, bandages, and the like. Such transdermal patches may be used to provide continuous or discontinuous infusion of CGRP in controlled amounts. The construction and use of transdermal patches for the delivery of pharmaceutical agents is well known in the art. See, for example, U.S. Pat. No. 5,023,252, the disclosure of which is herein incorporated by reference. Such patches may be constructed for continuous, pulsatile, or on-demand delivery of pharmaceutical. For example, a dose of CGRP or a pharmaceutically acceptable composition thereof may be combined with skin penetration enhancers including, but not limited to, oleic acid, oleyl alcohol, long chain fatty acids, propylene glycol, polyethylene glycol, isopropanol, ethoxydiglycol, sodium xylene sulfonate, ethanol, N-methylpyrrolidone, laurocapram, alkanecarboxylic acids, dimethylsulfoxide, polar lipids, N-methyl-2-pyrrolidone, and the like, which increase the permeability of the skin to the dose of CGRP and permit the dose of CGRP to penetrate through the skin and into the bloodstream. CGRP or a pharmaceutically acceptable composition thereof may be combined one or more agents including, but not limited to, alcohols, moisturizers, humectants, oils, emulsifiers, thickeners, thinners, surface active agents, fragrances, preservatives, antioxidants, vitamins, or minerals. CGRP or a pharmaceutically acceptable composition thereof may also be combined with a polymeric substance including, but not limited to, ethylcellulose, hydroxypropyl cellulose, ethylene/vinylacetate, polyvinyl pyrrolidone, and the like, to provide the composition in gel form, which may be dissolved in solvent such as methylene chloride, evaporated to the desired viscosity, and then applied to backing material to provide a patch. The backing can be any of the conventional materials such as polyethylene, ethyl-vinyl acetate copolymer, polyurethane and the like.

CGRP may also be administered transmucosally, i.e., to and across a mucosal surface. Transmucosal administration of a source of CGRP or a pharmaceutically acceptable composition thereof can be accomplished generally by contacting an intact mucous membrane with a source of CGRP or a pharmaceutically acceptable composition thereof, and maintaining the source in contact with the mucous membrane for a sufficient time period to induce the desired therapeutic effect. Preferably CGRP or a pharmaceutically acceptable composition thereof is administered to the oral or nasal mucosa such as the buccal mucosa, the sublingual mucosa, the sinuidal mucosa, the gum, or the inner lip. Particularly, the source of CGRP is any preparation usable in oral, nasal, sinuidal, rectal or vaginal cavities that can be formulated using conventional techniques well known in the art. For example, the preparation can be a buccal tablet, a sublingual tablet, a spray, and the like that dissolves or disintegrates, delivering drug into the mouth of the patient. A spray or drops can also be used to deliver the CGRP or a pharmaceutically acceptable composition thereof to nasal or sinuidal cavities. The preparation may or may not deliver the drug in a sustained fashion. Examples for manufacturing such preparations are disclosed, for example, in U.S. Pat. No. 4,764,378, which is specifically incorporated herein by reference. The preparation can also be a syrup that adheres to the mucous membrane. Suitable mucoadhesives include those well known in the art such as polyacrylic acids, preferably having the molecular weight between from about 450,000 to about 4,000,000, e.g. Carbopol™ 934P; sodium carboxymethylcellulose (NaCMC), hydroxypropylmethylcellulose (HPMC), e.g. Methocel™ K100, and hydroxypropylcellulose.

The transmucosal preparation can also be in the form of a bandages patch, and, the like that contains the drug and adheres to a mucosal surface. A mucoadhesive preparation is one that upon contact with intact mucous membrane, adheres to the mucous membrane for a sufficient time period to induce the desired therapeutic effect. Suitable transmucosal patches are described for example in PCT Publication WO 93/23011, which is specifically incorporated herein by reference. A suitable patch may comprise a backing which can be any flexible film that prevents bulk fluid flow and provides a barrier for to loss of the drug from the patch. The backing can be any conventional material such as polyethylene, ethyl-vinyl acetate copolymer, polyurethane and the like. In a patch involving a matrix which is not itself a mucoadhesive, the drug-containing matrix can be coupled with a mucoadhesive component (such as a mucoadhesive described above) in order that the patch may be retained on the mucosal surface. Suitable configurations include a patch or device wherein the matrix has a smaller periphery than the backing layer such that a portion of the backing layer extends outward from the periphery of the matrix. A mucoadhesive layer covers the outward extending portion of the backing layer such that the underside of the backing layer carries a layer of mucoadhesive around its periphery. The backing and the peripheral ring of mucoadhesive taken together form a reservoir which contains a drug-containing matrix (e.g. a tablet, gel or powder). It may be desirable to incorporate a barrier element between the matrix and the mucoadhesive in order to isolate the mucoadhesive from the matrix. The barrier element is preferably substantially impermeable to water and to the mucosal fluids that will be present at intended site of adhesion. A patch or device having such barrier element can be hydrated only through a surface that is in contact with the mucosa, and it is not hydrated via the reservoir. Such patches can be prepared by general methods well known to those skilled in the art. The preparation can also be a gel or film comprising a mucoadhesive matrix as described for example in PCT Publication WO 96/30013, which is specifically incorporated herein by reference.

The present invention also provides pharmaceutical kits for treating HF and/or improving renal function, comprising one or more containers comprising one or more CGRP pharmaceutical compositions of this invention. Such kits can also include additional drugs or therapeutics (e.g., antiproliferative or anti-clotting agents, or other compounds used to treat cardiovascular diseases and the like) for co-use with CGRP for treatment or prevention of HF and/or for improving renal failure. In this embodiment, the CGRP and the drug can be formulated in admixture in one container, or can be contained in separate containers for simultaneous or separate administration. The kit can further comprise a device(s) for administering the compounds and/or compositions, and written instructions in a form prescribed by a governmental agency regulating the manufacture, use or sale of pharmaceuticals or biological products, which instructions can also reflect approval by the agency of manufacture, use or sale for human administration.

The foregoing description is considered as illustrative only of the principles of the invention. Further, since numerous modifications and changes will be readily apparent to those skilled in the art, it is not desired to limit the invention to the exact construction and process shown as described above. Accordingly, all suitable modifications and equivalents may be resorted to falling within the scope of the invention as defined by the claims that follow.

The words “comprise,” “comprising,” “include,” “including,” and “includes” when used in this specification and in the following claims are intended to specify the presence of stated features, integers, components, or steps, but they do not preclude the presence or addition of one or more other features, integers, components, steps, or groups thereof.

We claim:

1. A method of treating heart failure and/or renal failure in a patient, comprising administering CGRP to said patient at a rate between about 50 and 500 ng/min for a time between 30 minutes and 8 hours per day as needed to provide symptomatic relief, prevent exacerbation of symptoms, and/or prevent and/or delay progression of the disease state of heart failure in said patient.

2. The method of claim 1, wherein said CGRP is administered parenterally, orally, sublingually, intranasally, intracoronary, intra-arterially, intravenously, transmucosally, or intradermally.

3. The method of claim 1, wherein said CGRP is administered via a constant rate pump, a variable rate pump, a programmable pump, or an osmotic pump.

4. The method of claim 1, wherein said CGRP is administered transdermally.

5. The method of claim 4, wherein said transdermal administration is accomplished with a transdermal delivery device, a cream, an ointment, a patch or a bandage.

6. The method of claim 4, wherein said CGRP is combined with a penetration enhancer.

7. The method of claim 6, wherein said penetration enhancer is selected from the group consisting of propylene glycol, polyethylene glycol, isopropanol, oleyl alcohol, ethoxydiglycol, sodium xylene sulfonate, ethanol, oleic acid, N-methylpyrrolidone, laurocapram, alkanecarboxylic acids, dimethylsulfoxide, polar lipids, and N-methyl-2-pyrrolidone.

8. The method of claim 6, wherein said penetration enhancer is oleic acid, oleyl alcohol or a long-chain fatty acid.

9. The method of claim 1, wherein said CGRP is combined with one or more agents selected from the group consisting of alcohols, moisturizers, humectants, oils, emulsifiers, thickeners, thinners, surface active agents, fragrances, preservatives, antioxidants, vitamins, and minerals.

10. The method of claim 1, further comprising administering at least one drug selected from the group consisting of anti-proliferative agents, anti-clotting agents, vasodilators, diuretics, beta-blockers, calcium ion channel blockers, blood thinners, cardiotonics, ACE inhibitors, anti-inflammatories, and antioxidants.

11. The method of claim 10, wherein said CGRP and said at least one drug are administered as an admixture, separately and simultaneously, or separately in any order.

12. The method of claim 1, wherein the length of said treatment is sufficient to improve renal blood flow, glomerular filtration rates, and/or serum levels of urea and creatinine in said patient.

13. The method of claim 1, wherein said treatment is administered to said patient in a hospital for the duration of the time said patient is in the hospital.

14. The method of claim 1, wherein said treatment is administered to a patient on an outpatient basis.

15. The method of claim 1, wherein said patient is a pediatric patient.

16. The method of claim 1, wherein said treatment is followed by maintenance therapy comprising administration of CGRP a rate between 0.8 to 10 ng/kg/min as needed to relieve or prevent exacerbation of symptoms, or prevent or delay progression of said heart failure.

17. A method of treating heart failure in a patient, comprising administering CGRP to said patient at a rate between about 500 and 600 ng/min for up to 8 hours per day for at least three consecutive days or several times per week as needed to provide symptomatic relief, prevent exacerbation of symptoms, and/or prevent and/or delay progression of the disease state of heart failure in said patient.

18. The method of claim 17, wherein said treatment is provided as outpatient therapy, in in emergency room, or in an intensive care unit.

19. The method of claim 17, wherein said treatment further improves the quality of life of said patient.

20. A method of treating heart failure and/or renal failure in a patent, comprising administering CGRP to a heart failure patient as an initial or maintenance therapy at a rate between 0.8 to 10 ng/kg/min two or more times per day as needed to provide symptomatic relief, prevent exacerbation of symptoms, and/or prevent and/or delay progression of the disease state of heart failure in said patient.

21. A method of preventing or reducing the risk of occurrence of myocardial infarction, comprising administering to a human at risk of having a myocardial infarction a CGRP formulation in an amount effective to prevent or reduce the risk of said myocardial infarction.

22. A method of counteracting ischemia in a patient, wherein said ischemia is due to a myocardial infarction, said method comprising administering CGRP to said patient as an initial or maintenance therapy, alone or in conjunction with other interventional therapies, at a rate between 0.8 to 16 ng/kg/min for up to 24 hours per day as needed to provide cardio protection, reduction in infarction size, reduction in reperfusion injury, symptomatic relief, and/or prevent exacerbation of symptoms.
